(19) United States
(12) Patent Application Publication
(10) Pub. No.: US 2023/0248851 A1
(43) Pub. Date: Aug. 10, 2023

(54) PROCEDURE FOR OBTAINING GADOTERATE MEGLUMINE FROM HIGH-PURITY TETRAXETAN (DOTA) AND ITS USE IN THE PREPARATION OF INJECTABLE GALENICAL FORMULATIONS

(71) Applicant: JUSTESA IMAGEN S.A.U., Coslada, Madrid (ES)

(72) Inventors: Ignacio ALONSO SILVA, Coslada, Madrid (ES); Juan Andrés CERECEDA PÉREZ, Coslada, Madrid (ES); Felix Ramón MARTÍN MARTÍN, Coslada, Madrid (ES); Maria Ángeles REGUEIRA GÓMEZ, Coslada, Madrid (ES); Roberto CHICHARRO MARTÍN, Coslada, Madrid (ES); ÁIvaro GARCÍA RAMOS, Coslada, Madrid (ES); Irene GARCÍA SALADO, Coslada, Madrid (ES); Vicente MONTIEL LEGUEY, Coslada, Madrid (ES); Eduardo EXPÓSITO RODRÍGUEZ, Coslada, Madrid (ES); Vicente GARCÍA GARCÍA, Coslada, Madrid (ES); David VALERO VALERO, Coslada, Madrid (ES); Alfonso SÁEZ FÉRNANDEZ, Coslada, Madrid (ES); Francisco GALLUD MARTINÉZ, Coslada, Madrid (ES)

(21) Appl. No.: 18/009,702

(22) PCT Filed: Jun. 10, 2021
(86) PCT No.: PCT/EP2021/065599

§ 371 Date: Dec. 9, 2022

(30) Foreign Application Priority Data
Jun. 10, 2020 (EP) 20382500.5

(51) Int. Cl. A61K 49/10; C07D 257/02

(52) U.S. Cl. CPC A61K 49/108; C07D 257/02

(57) ABSTRACT

The present invention refers to a process for obtaining gadoterate meglumine from high purity tetraxetan (DOTA) which does not require the use of organic solvents and optimizes the conditions of the synthetic process. The tetraxetan from which the gadoterate meglumine is obtained by a synthetic process that includes a purification step in which at least one electrodialysis is performed. This procedure makes it possible to obtain a tetraxetan and a gadoterate meglumine with minimal amounts of impurities.

### FIELD OF THE INVENTION

The present invention relates to a process for obtaining gadoterate meglumine from high purity tetraxetan which does not require the use of organic solvents and optimizes the conditions of the synthesis. Gadoterate meglumine is used as a contrast agent in diagnostic tests. Therefore, the present invention can be included in the field of pharmacology or pharmaceutical chemistry.

### BACKGROUND OF THE INVENTION

Diagnostic imaging is a technique widely used in the field of medicine for the visualization of biological processes, organs or tissues, which requires the use of contrast media. One type of these contrast media is gadolinium-based derivatives, which are compounds increasingly used in magnetic resonance imaging (MRI) studies. However, the toxicity of linear gadolinium-based contrast agents and the existence of free gadolinium deposits in certain areas of the brain have caused that already existing, but not widely used, macrocyclic derivatives arise as a safer alternative and with less accumulation of free gadolinium. Among the macrocyclic derivatives, gadoterate meglumine is the compound with the least accumulation of gadolinium in the brain, possibly due to its high stability (Am. J. Neuroradiol. 2016, 37, 1192-1198).

The synthetic process to obtain tetraxetan, a precursor of gadoterate meglumine, involves tedious and expensive purification processes that, in most cases, use, among others, ion exchange resins that require subsequent treatment with organic solvents to desorb the compound of interest. U.S. Pat. No. 5,922,862 describes the purification of tetraxetan and its derivatives by elution through PVP resin. U.S. Pat. No. 5,334,729 discloses the purification of these complexes by using cation exchange columns.

EP3223863 and EP2799090 describe the final formulation of a 0.5 M solution of gadoterate meglumine in water for injections from isolated tetraxetan, gadolinium oxide and 35 meglumine. This process involves successive and long adjustment steps of tetraxetan and gadolinium oxide amounts, so that a tetraxetan content between 0% and 0.25% remains. Then, there is another pH adjustment step with meglumine and finally a concentration adjustment to obtain a 0.5 M solution, which is used as a contrast medium.

WO2017/103258 describes a method for the synthesis of DOTA comprising steps of crystallizing and filtering using membrane filtration, although in both steps methanol is added, remaining a content of methanol of ca. 4% wt. in the final product.

Therefore, there is a need to optimize the tetraxetan synthetic process to reduce the number of steps to obtain high-purity final product. In parallel, it is necessary to minimize the degradation of reagents and intermediates during the process and eliminate the use of organic solvents in order to reduce the environmental impact and possible impurities in the final product.

### DESCRIPTION OF THE INVENTION

The present invention solves the drawbacks of the procedures of the state of the art optimizing the synthetic process to obtain tetraxetan by using milder reaction conditions leading to lower degradation of the starting reagents, and carrying out a combination of purification and isolation techniques which allow obtaining high purity tetraxetan without the use of organic solvents (green chemistry). From this tetraxetan, gadoterate meglumine is obtained, an API which is used with a simple process of dissolution and concentration adjustment in the preparation of aqueous formulations for injections for subsequent use in magnetic resonance imaging diagnosis. The present invention greatly simplifies the process for obtaining gadoterate meglumine by optimizing the manufacturing process and isolating the gadoterate meglumine as a solid which meets well-defined specifications and that, due to the use of high purity tetraxetan, does not require any additional purification process.

Therefore, in a first aspect the present invention refers to a procedure to obtain tetraxetan comprising the following steps:
(a) obtaining of tetraxetan by the reaction described below:

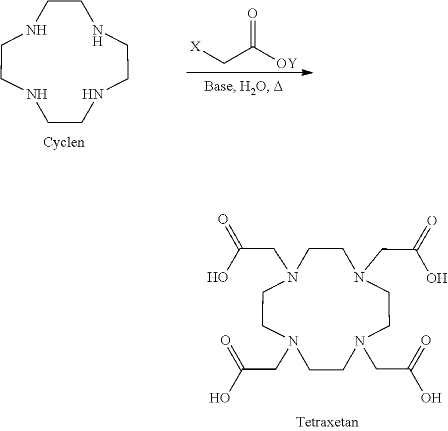

where X is a halogen, preferably chlorine, Y is selected from hydrogen or an alkaline element, preferably sodium, the base is selected from potassium, sodium or lithium hydroxide, preferably sodium hydroxide, the pH is maintained between 7 and 8.5, preferably 8 and the reaction temperature is kept between 70 and 100° C., preferably 80° C., and crystallization of the obtained tetraxetan by lowering the pH below 3 to obtain a tetraxetan crude;
(b) purification of the crystallized tetraxetan crude obtained in the previous step, involving at least one electrodialysis;
(c) isolation of the product obtained in the previous step, preferably by spray drying.

In the present invention, tetraxetan is also referred as DOTA.

In a preferred embodiment, the crystallization of the tetraxetan obtained in the reaction of step (a) is carried out at a pH below 3, preferably equal or below 2 and more preferably equal or below 1. This decrease of pH may be carried out by adding an acid commonly used, such as HCl. These pH values allow a good yield since they favour the transformation of all carboxylate groups to carboxylic acids and avoid the use of organic solvents as no purification is necessary. Variations of pH of ±0.2 to ±0.5 are considered within the scope of the invention and may be applied to the described pH values.

In a preferred embodiment, cationic, anionic, bipolar membranes or combinations thereof are used in electrodialysis.

In another preferred embodiment, two consecutive electrodialysis are performed in the purification step (b). In a first more preferred embodiment, the first electrodialysis is performed using:
a combination of cationic, anionic and bipolar membranes or
a combination of cationic and anionic membranes where preferably the anionic membranes are monoselective.

In another, more preferred procedure, the second electrodialysis is carried out using:
a combination of cationic, anionic and bipolar membranes or
a combination of cationic and bipolar membranes.

Preferably, in both electrodialysis of this preferred embodiment, the pH is maintained between 2 and 6.

In another preferred embodiment, in the purification step (b), a nanofiltration with constant volume is performed prior to electrodialysis. In a more preferred embodiment, the electrodialysis is performed using:
a combination of cationic, anionic and bipolar membranes or
a combination of cationic and anionic membranes where preferably the anionic membranes are monoselective or
a combination of cationic and bipolar membranes.

Preferably, the pH during nanofiltration is maintained between 2 and 8, more preferably between 3 and 5 and even more preferably at 4.

Preferably, the pH during electrodialysis of this preferred embodiment is kept between 2 and 5, more preferably at 4.

In a preferred embodiment, in step (c) the compound is isolated by spray drying in which the temperature of the inlet air is 160-200° C. In a more preferred embodiment, the inlet air temperature is 170-190° C. In a more preferred embodiment, the inlet air temperature is 175-185° C. In an even more preferred embodiment, the inlet air temperature is 180° C. In a preferred embodiment, the outlet air temperature is 90-120° C. In a more preferred embodiment, the outlet air temperature is 105-115° C. In an even more preferred embodiment, the outlet air temperature is 110° C.

The spray-drying step involves a fundamental improvement of the process of the invention in respect to other similar process known in the art. Due to the high-purity of DOTA obtained in step (b) there is no need to use organic solvents to isolate the product as described in, for example, WO2017/103258 where high amounts of methanol are used in several steps, and the final product presented traces of said methanol. Therefore, by applying the process of the present invention the final product is free of organic solvents, something that is advantageous for the manufacturing of a pharmaceutical product.

In another preferred embodiment, the product obtained in step (c) is characterised by having a maximum residual amount of the alkaline element, preferably sodium, of 500 ppm (0.05%) and a maximum residual amount of halide, preferably chloride, of 500 ppm (0.05%).

Another aspect of the invention relates to tetraxetan obtained by the process described 35 above, characterised by having (a) a maximum residual amount of the alkaline element, preferably sodium, determined for example by inductively coupled plasma mass spectrometry (ICP-MS), of 500 ppm (0.05%), preferably 100 ppm (0.01%), more preferably 50 ppm (0.005%), (b) a maximum residual amount of halide, preferably chloride, determined, for instance, by inductively coupled plasma mass spectrometry (ICP-MS), of 500 ppm (0.05%), preferably 100 ppm (0.01%), more preferably 50 ppm (0.005%), even more preferably 20 ppm (0.002%), and (c) a maximum residual amount of solvents and volatile substances below the detection limit.

In the present invention, “limit of detection” (LOD) or “detection limit” is defined as the lowest quantity of an analyte whose signal can be distinguished from the absence of that substance (“noise”) with a stated confidence level and is usually defined as the minimum amount or concentration of substance that can be reliably detected by a given analytical method. In practice, the limit of detection would be the minimum concentration obtained from the analysis of a sample (containing the analyte) that can be discriminated from the concentration obtained from the determination of a blank sample, i.e., a sample with no analyte present. In this respect, the limit of detection of the possible solvents used in the procedure of this invention can be set at 10 ppm (0.001%) determined by means of e.g., gas chromatography/mass spectrometry (GC-MS).

The procedure of the present invention allows obtaining high yields and/or higher levels of purity than the tetraxetan obtained by means of previously described procedures in the state of the art. The purity of the tetraxetan obtained by the process described in the present invention is typically at least over 50%, at least over 60%, at least over 70%, at least over 80%, at least over 90% or more. The purity of the obtained tetraxetan can be measured by methods known to anyone skilled in the art, such as inductively coupled plasma mass spectrometry (ICP-MS), high performance liquid chromatography (HPLC) or gas chromatography/mass spectrometry (GC-MS), as will be shown in the examples.

The term “halogen” relates to chlorine (CI), bromine (Br) and iodine (I). In their anionic form, they are mentioned as halides.

The term “alkaline” means lithium (Li), sodium (Na) and potassium (K).

The term “base” means a substance which when dissolved in an aqueous medium releases hydroxyl ions (OH—) and gives to the medium alkaline properties. Preferably, it refers to alkaline hydroxides such as potassium hydroxide (KOH), sodium hydroxide (NaOH) and lithium hydroxide (LiOH).

The term “electrodialysis” relates to a membrane process in which ions are transported through an ion exchange membrane by an electrical field as driving force. The electrodialysis system consists of an electrolyte solution, a concentrate solution and a diluate solution. In the present invention, “electrolyte solution” refers to a solution of sulfuric acid at pH=1-3 or to a diluted solution of sodium sulfate. In the present invention, “concentrate solution” refers to a diluted solution of sulfuric acid in water. In the present invention, “diluate solution” refers to a solution of tetraxetan. The electrodialysis procedure of the invention can be carried out with different configurations for industrial scale-up. Non-limiting examples of possible configurations are shown in FIGS. 1 and 2.

In the present invention, a control of the base addition is made in order to minimise the formation of salts in the reaction crude. Additionally, the treatment of the reaction crude with electrodialysis and/or nanofiltration allows the removal of inorganic ions in addition to other impurities present in the reaction crude, such as organic impurities.

Another aspect of the invention refers to a process to obtain gadoterate meglumine, which comprises the steps of the process to obtain tetraxetan as described above and the following additional steps:
(d) reaction of the product obtained in step (c) with a gadolinium derivative, preferably Gd₂O₃, and meglumine,
(e) isolation of the product obtained in step (d) by spray drying.

In the present invention, the high quality DOTA obtained in the previous steps (a)-(c) allows directly isolating the reaction mass of step (d) on solution by spray drying step without any purification step required. This fact increases yields and eliminates the need to use organic solvents, compared to other techniques of the art in which meglumine gadoterate is isolated from the solution a slurry by crystallization with organic solvents.

To a tetraxetan solution, previously purified by any of the above-described techniques, the corresponding gadolinium derivative (1 eq.) is added. The mixture is heated for a certain time. The pH of the reaction is kept constant during the whole process by adding of meglumine together with the gadolinium derivative in step (d). This improves the reaction times since it generates meglumine gadoterate directly and prevents the formation of highly water insoluble gadolinium hydroxides.

In a preferred embodiment, the initial tetraxetan concentration is 50-250 g/L.

In the present invention, “gadolinium derivative” refers to gadolinium oxide or a gadolinium salt, such as gadolinium chloride, gadolinium sulfate, etc. Preferably, the gadolinium derivative is gadolinium oxide.

In a preferred embodiment, the pH of the reaction remains constant. In a preferred embodiment, the pH of the reaction is between pH=1-7. In a more preferred embodiment, the pH of the reaction is between pH=2-5. In an even more preferred embodiment, the pH of the reaction is between pH=3.5-4.5. In a preferred embodiment, the pH of the reaction is maintained by addition of meglumine.

In a preferred embodiment, the solvent is water.

In a preferred embodiment, the temperature of the reaction is between 80-100° C. In a more preferred embodiment, the temperature of the reaction is 85° C.

In a preferred embodiment, the final pH of the solution is adjusted to a pH value that is between 6.5-8 by adding meglumine. In another preferred embodiment, the final pH of the solution is adjusted to a pH value that is between 6.5 and 7.5 by the addition of meglumine.

In a preferred embodiment, in step (e) the compound is isolated by spray drying. During the process the temperature of the inlet air is between 160-200° C. In a more preferred embodiment, the inlet air temperature is between 170-190° C. In a more preferred embodiment, the inlet air temperature is between 175-185° C. In an even more preferred embodiment, the inlet air temperature is 180° C. In another preferred embodiment, the outlet air temperature is between 90-120° C. In a more preferred embodiment, the outlet air temperature is between 105-115° C. In an even more preferred embodiment, the outlet air temperature is 110° C.

In a preferred embodiment, the product obtained in step (d) is subjected to at least one ultrafiltration.

The gadoterate meglumine obtained in this way does not require any further purification process, so it can be directly used to prepare pharmaceutical compositions.

Another aspect of the invention regards gadoterate meglumine obtained by the process according to the claim characterized by a maximum amount of residual solvents and other volatile substances below the detection limit. The detection limit of the solvents (which may be, for example, those mentioned above) is approximately 10 ppm (0.001%). For other related substances such as cyclen, chloroacetic acid, glycolic acid, etc. it is approximately 0.001%, determined by e.g. high performance liquid chromatography (HPLC).

Another aspect of the invention relates to a pharmaceutical composition comprising gadoterate meglumine as described above, preferably such a pharmaceutical composition is a contrast agent formulated as injectable.

To obtain this pharmaceutical composition, procedures and techniques known to anyone skilled in the art and well established by the applicable pharmacopoeia shall be used. For example, in the case of injectables, the gadoterate meglumine will be dissolved in water for injection according to the desired concentration and the corresponding treatment will be carried out to finally obtain the vials ready for use.

### BRIEF DESCRIPTION OF THE FIGURES

FIG. 1.—Shows the scheme of the tetraxetan purification process by means of two electrodialysis.

FIG. 2.—Shows the scheme of the tetraxetan purification process by means of nanofiltration followed by electrodialysis.

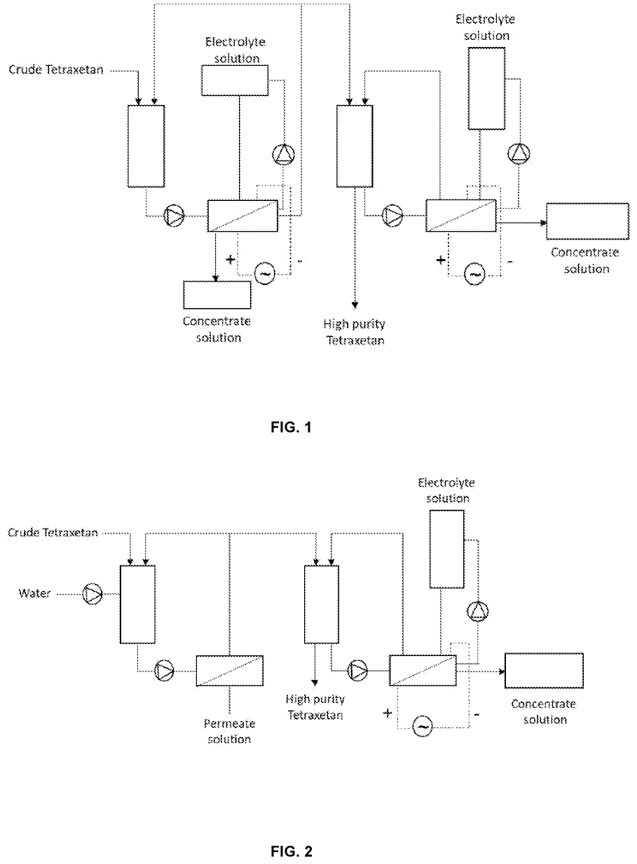

### EXAMPLES OF THE INVENTION

### Example 1. Synthesis of Raw Tetraxetan

A 125 g/L solution of cyclen (1 eq.) in water is prepared. Then chloroacetic acid (4.5 eq.) is added. The mixture is heated to 80° C. and the pH of the reaction is adjusted to 8, adding sodium hydroxide using a pH controller. When the reaction is completed, the pH is raised to 10 maintaining the temperature for the necessary time. The reaction mixture is then cooled to 65° C. and then concentrated HCl is added until pH<1. Finally, the solvent is partially removed under reduced pressure, the mixture is cooled and the tetraxetan crude obtained is centrifuged (80-85% yield).

### Example 2. Tetraxetan Purification by a Combination of Nanofiltration and/or Electrodialysis Techniques

A. Treatment of Raw Tetraxetan by Means of Two Electrodialysis:

A solution of raw tetraxetan (45 g/L) is purified by a first electrodialysis containing monoselective anionic membranes and cationic membranes. The pH of the diluate solution is maintained between 2-5. The concentrate and electrolyte solutions can be sulfuric acid at pH=1−3 or sodium sulfate ata concentration of 5 g/L (95% yield; Cl<0.01%; Na<0.5%).

This electrodialysed solution is then purified by a second electrodialysis containing cationic and bipolar membranes. The pH of the diluate solution is maintained between pH=2.8-4.5. The concentrate and electrolyte solutions are as described above. In this way, a tetraxetan solution is obtained which is directly used in the next isolation step (90-95% yield; Na<0.01%).

B. Treatment of Raw Tetraxetan by Means of Nanofiltration and Electrodialysis:

A 45 g/L solution of raw tetraxetan is prepared and the pH is adjusted to 4 by adding sodium hydroxide solution. This solution is passed through the nanofiltration membrane, resulting in two streams, the rejection solution, which returns to the initial solution containing tetraxetan, and the permeate solution, which is collected in a different tank. This last solution contains inorganic ions and low molecular weight organic impurities. The volume of the rejected solution is kept constant by the addition of water. The nanofiltration process ends when the concentration of chloride anions is below the value of the specification (98-99% yield; Cl<0.01%; Na<4.1%)

This tetraxetan solution at a concentration of 45 g/L is then subjected to an electrodialysis process in which cationic and anionic membranes are used, and the pH of the diluate solution is maintained between pH=2.8-4.5. Or, a tetraxetan solution is treated with cationic membranes and monoselective anionic membranes, and the pH of the diluate solution is maintained between pH=2-3. Or, a tetraxetan solution at a concentration of 45 g/L is treated with cationic and bipolar membranes, and the pH of the diluate solution is maintained at pH=4. The electrolyte and concentrate solutions are a sulfuric acid or sodium sulfate solution. The electrodialysis process ends when the concentration of sodium cations is less than the specification value. In this way, a tetraxetan solution is obtained which goes directly to the isolation step (90-95% yield; Na<0.008%).

### Example 4. Isolation and Characterization of Tetraxetan

The solution from the purification process can be used in the synthesis of gadoterate meglumine or, alternatively, tetraxetan can be isolated. Tetraxetan can be isolated from the aqueous solution by spray drying at an inlet air temperature of 180° C. and an outlet air temperature of 110° C. The result is a product that meets the specifications described below (97-99% yield; Na<0.008%).

The high purity tetraxetan obtained meets the following specifications:
Appearance: white powder
Identification:
   IR spectrum: comparable with the reference tetraxetan
   HPLC: retention time matching that of the reference
Water≤8.5%
Clear solution, no more coloured than reference solution Y7
Sodium≤500 ppm (0.05%)
Halides≤500 ppm (0.05%)
Residue on ignition≤0.10%
Related impurities:
   DO3A≤0.05%
   Any other individual impurity 0.05%
   Total impurities 0.5%
Titration (acid-base): 98.0-102.0% on dry product
Bacterial Endotoxin≤22 EU/g
Total aerobic microbial count≤10² CFU/g
Total combined yeast and mould count≤10² CFU/g
Escherichia coli: absence/1 g
Residual solvents<10 ppm (0.001%)

### Example 5. Gadoterate Meglumine Synthesis

To a solution of tetraxetan in water (200 g/L), Gd₂O₃ (1 eq.) is added. The mixture is heated 25 to 85° C. for 2-4 h. The pH of the reaction is maintained at pH=4 by adding meglumine. The reaction ends when the final amount of tetraxetan and free gadolinium is less than 0.005% (w/v). The pH of the solution is then raised between pH=6.5−8 by the addition of meglumine (quantitative yield)

### Example 6. Isolation and Characterization of Gadoterate Meglumine

The final product dissolved in water is subjected to depyrogenizing ultrafiltration and then isolated by spray drying. The working conditions are as follows, inlet air temperature between 175-185° C. and outlet air temperature 110° C. (97-98% yield). The gadoterate meglumine isolated by this procedure has the following specifications:
Appearance: white powder
Water≤10.0%
Identification:
   HPLC: retention time similar to that of the reference
   IR spectrum: similar to the reference
Clear or no more opalescent solution than European Pharmacopoeia and American Pharmacopoeia (USP) reference solution I for the clarity and opalescence test of solutions
Colour of the solution≤Y7
pH=6.5−8
Related impurities:
   DO3A≤0.05%
   Tetraxetan≤0.1%
   Any other individual impurity≤0.05%
   Total impurities≤0.50%
Purity (HPLC): 97-103% on anhydrous product
Meglumine content: 24.5-26.5% on anhydrous product
Free Gd content≤0.01%
Total Gd content: 19-22%.
Foreign matter≤100 ppm (0.01%)
Bacterial endotoxin<20UE/g
Total aerobic microbial count (TAMC)≤10² CFU/g
Total combined yeast and mould count (TYMC)≤10² CFU/g
Escherichia coli: absence
Residual solvents<10 ppm (0.001%)
Elemental impurities:
   As≤1500 ppb
   Pb≤500 ppb
   Cd≤200 ppb
   Hg≤300 ppb
   V≤1000 ppb
   Ni≤2000 ppb
   Co≤500 ppb
   Pd≤1000 ppb
   Cu≤30000 ppb
   Li≤25000 ppb
   Sb≤9000 ppb

### Example 7. Galenic Formulation of Gadoterate Meglumine

With the obtained gadoterate meglumine, injectable galenic formulations were prepared to be used in magnetic resonance imaging diagnosis.

TABLE 1
Solution of gadoterate meglumine in
water at a concentration of 0.5M.

| | | |
|---|---|---|
| Gadoterate meglumine | 1 g | 0.5M |
| WFI* up to a total volume of | 2.65 mL | |

*WFI, water for injection.

TABLE 2
Solution of 0.5M gadoterate meglumine
and tetraxetan as excipient in water.

| | | | |
|---|---|---|---|
| Gadoterate meglumine | 1 | g | 0.5M |
| WFI up to a volume of | 2.65 | mL | |
| Tetraxetan (excipient) | 0.133-1.33 | mg | 0.025-0.25% (mol/mol) |

These two prepared formulations with the gadoterate meglumine isolated from the present invention are depyrogenized and sterilized. These two formulations meet the following specifications:
Clear solution, no more coloured than the reference solution Y7 of the European pharmacopoeia and the American pharmacopoeia (USP) for the test of clarity and opalescence of solutions
Identification:
   IR spectrum: similar to the reference compound
   HPLC: retention time similar to the reference standard.
pH=6.9-8.
Density=1.1649−1.1828 g/mL
Free of visible particles.
Subvisible particles<6000 (particle size<10 μm); <600 (particle size<25 μm)
Absorbance: at 450 nm<0.40AU; at 500 nm<0.20AU
Assay:
   Gadoterate: 26.53-29.33% (w/v)
   Total gadolinium: 7.63-8.10% (w/v)
   Free gadolinium<0.005% (w/v)
   Meglumine: 9.27-10.25% (w/v)
Related impurities:
   DO3A<0.02% (w/v)
   Tetraxetan<0.05% (w/v)
   Any other impurity<0.02% (w/v)
   Total impurities<0.2% (w/v)
Elemental impurities:
   As≤560 ppb
   Pb≤190 ppb
   Cd≤75 ppb
   Hg≤110 ppb
   V≤370 ppb
   Ni≤750 ppb
   Co≤190 ppb
   Pd≤370 ppb
   Cu≤1100 ppb
   Li≤940 ppb
   Sb≤3400 ppb
Sterile solution, free of bacterial endotoxins (<8EU/mL).

### Example 8. Comparison of Tetraxetan Obtained by the Process of the Invention

A comparison between the levels of present impurities in tetraxetan samples obtained by the process of the invention (DOTA 001-003) and other commercially available tetraxetan samples from different suppliers (Supplier 1-5) was made. The data are summarized in the following tables:

TABLE 3
Determination of elemental alkaline and halide impurities in tetraxetan
by inductively coupled plasma mass spectrometry (ICP-MS)

| | Na | K | Cl | Br |
|---|---|---|---|---|
| | (ppm) | (ppm) | (ppm) | (ppm) |
| | (LOD | (LOD | (LOD | (LOD |
| SAMPLE | 1 ppm) | 1 ppm) | 2 ppm) | 10 ppm) |
| Supplier 1 | 62 | 7 | 60 | n.d. |
| Supplier 2 | 68 | n.d. | 68 | n.d. |
| Supplier 3 | 82 | n.d. | 121 | n.d. |
| Supplier 4 | 20550 | 500 | 8500 | 49000 |
| Supplier 5 | 1500 | 150 | 50 | n.d. |
| DOTA-001 | 45 | n.d. | 2 | n.d. |
| DOTA-002 | 59 | n.d. | 12 | n.d. |
| DOTA-003 | 47 | n.d. | 13 | n.d. |

TABLE 4
Determination of residual solvents in tetraxetan by
gas chromatography/massspectrometry (GC-MS)

| | | Ethyl | | | | Iso- |
|---|---|---|---|---|---|---|
| | Ethanol | acetate | Acetone | Ammonia | Methanol | propanol |
| | (ppm) | (ppm) | (ppm) | (ppm) | (ppm) | (ppm) |
| | (LOD10 | (LOD10 | (LOD10 | (LOD10 | (LOD10 | (LOD10 |
| SAMPLE | ppm) | ppm) | ppm) | ppm) | ppm) | ppm) |
| Supplier 1 | 395 | n.d. | 20 | | | n.d. |
| Supplier 2 | n.d. | 2 | 44 | | | n.d. |
| Supplier 3 | n.d. | n.d. | 10 | | 25 | n.d. |
| Supplier 4 | 75 | n.d. | n.d. | | | n.d. |
| Supplier 5 | 1500 | n.d. | n.d. | 67000 | | 160 |
| DOTA-001 | n.d. | n.d. | n.d. | n.d. | n.d. | n.d. |
| DOTA-002 | n.d. | n.d. | n.d. | n.d. | n.d. | n.d. |
| DOTA-003 | n.d | n.d. | n.d | n.d. | n.d. | n.d. |

TABLE 5
Tetraxetan purity determined by HPLC for each sample

| SAMPLE | DOTA (%) |
|---|---|
| Supplier 1 | 99.88 |
| Supplier 2 | 99.85 |
| Supplier 3 | 99.86 |
| Supplier 4 | 90.81 |
| Supplier 5 | 96.30 |
| DOTA-001 | 99.99 |
| DOTA-002 | 99.99 |
| DOTA-003 | 99.99 |

TABLE 6
Determination of other impurities by high performance liquid
chromatography (HPLC)

| | | | Chloroacetic | | Any other | |
|---|---|---|---|---|---|---|
| | CYCLEN | DO3A | acid | Glycolic acid | individual | Total |
| | (%) | (%) | (%) | (%) | impurity (%) | impurities |
| | (LOD | (LOD | (LOD | (LOD | (LOD | (%) (LOD |
| SAMPLE | 0.001%) | 0.001%) | 0.001%) | 0.001%) | 0.001%) | 0.001%) |
| Supplier 1 | 0.006 | n.d. | 0.083 | 0.005 | 0.035 | 0.117 |
| Supplier 2 | 0.006 | 0.009 | 0.080 | n.d. | 0.057 | 0.152 |
| Supplier 3 | 0.009 | n.d. | 0.084 | 0.011 | 0.016 | 0.100 |
| Supplier 4 | n.d. | 0.003 | n.d. | n.d. | 0.041 | 0.190 |
| Supplier 5 | n.d. | 0.260 | n.d. | n.d. | n.d. | 0.420 |
| DOTA-001 | n.d. | 0.004 | n.d. | n.d. | 0.004 | 0.008 |
| DOTA-002 | n.d. | 0.006 | n.d. | n.d. | 0.003 | 0.009 |
| DOTA-003 | n.d. | n.d. | n.d. | n.d. | 0.009 | 0.009 |

1. A process to obtain tetraxetan that comprises the following steps:
(a) obtention of tetraxetan by the following reaction: 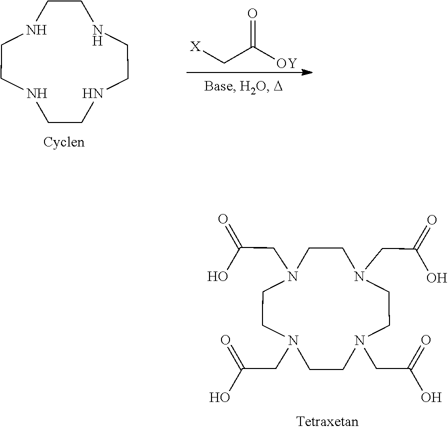
where X is a halogen, Y is selected from hydrogen or an alkaline element, the base is selected from potassium, sodium or lithium hydroxide, the pH is maintained between 7 and 8.5 and the reaction temperature is maintained between 70 and 100° C., and crystallization of the obtained tetraxetan by lowering the pH below 3 to obtain a tetraxetan crude;
(b) purification of the crystallized tetraxetan crude obtained in the previous step, involving at least one electrodialysis;
(c) isolation of the product obtained in the previous step by spray drying.

2. The process according to claim 1, where X is chlorine and/or Y is sodium.

3. The process according to any one of the claim 1 or 2, where the base is sodium hydroxide.

4. The process according to any one of the claims 1 to 3, where the pH of the reaction is maintained at 8 and/or the temperature is maintained at 80° C.

5. The process according to any one of the claims 1 to 4, where the pH in the crystallization of step (a) is below 2.

6. The process according to claim 5, where the pH in the crystallization of step (a) is below 1.

7. The process according to any one of claims 1 to 6, where cationic, anionic, bipolar membranes or combinations thereof are used in electrodialysis.

8. The process according to any of the claims 1 to 7, where in the step (b) of purification two consecutive electrodialysis are performed.

9. The process according to claim 8, where the first of the two electrodialysis is carried out using:
a combination of cationic, anionic and bipolar membranes or
a combination of cationic and anionic membranes, where preferably the anionic membranes are monoselective.

10. The process according to any of the claim 8 or 9, where the second electrodialysis is carried out using:
a combination of cationic, anionic and bipolar membranes or
a combination of cationic and bipolar membranes.

11. The process according to any of the claims 8 to 10, where in both electrodialysis the pH is maintained between 2 and 6.

12. The process according to any of the claims 1 to 7, where in the step (b) of purification a nanofiltration at constant volume is performed prior to electrodialysis.

13. The process according to claim 12, where electrodialysis is carried out using:
a combination of cationic, anionic and bipolar membranes or
a combination of cationic and anionic membranes, where preferably the anionic membranes are monoselective or
a combination of cationic and bipolar membranes.

14. The process according to any of the claim 12 or 13, where the pH during nanofiltration is kept between 2 and 8.

15. The process according to claim 14, where the pH during nanofiltration is kept between 3 and 5.

16. The process according to claim 15, where the pH during nanofiltration is kept at 4.

17. The process according to any of the claims 12 to 16, where the pH during electrodialysis is kept between 2 and 5.

18. The process according to claim 17, where the pH during electrodialysis is kept at 4.

19. Tetraxetan obtained by the process of any of the claims 1 to 18, characterized by a maximum residual amount of the alkali element, preferably sodium, of 500 ppm (0.05%), a maximum residual amount of halide, preferably chloride, of 500 ppm (0.05%) and a maximum residual amount of solvents and volatile substances below the detection limit.

20. A process to obtain gadoterate meglumine comprising the steps of the process according to claims 1 to 18 and the following additional steps:
(d) reaction of the product obtained in step (c) with Gd₂O₃ and meglumine
(e) isolation of the product obtained in step (d) by spray drying.

21. The process according to claim 20, in which in step (e) the isolation of the compound is carried out by spray drying in which the temperature of the inlet air to the spray drying system is 160-200° C., and/or the temperature of the outlet air is 90-120° C., preferably

22. The process according to claim 21, in which the temperature of the inlet air to the spray drying system is 170-190° C., preferably 175-185° C., more preferably 180° C., and/or the temperature of the outlet air is 105-115° C., preferably 110° C.

23. Gadoterate meglumine obtained by the process of either claims 20 to 22, characterized by a maximum amount of residual solvents and other volatile substances below the detection limit.

24. Pharmaceutical composition comprising gadoterate meglumine according to claim 23.

25. Pharmaceutical composition according to claim 24, which is a contrast agent formulated as injectable.
